(19) United States
(12) Patent Application Publication
(10) Pub. No.: US 2002/0006969 A1
(43) Pub. Date: Jan. 17, 2002

(54) System and method for converting light hydrocarbons into heavier hydrocarbons and for treating contaminated water

(76) Inventors: John Timothy O'Beck, Tulsa, OK (US); Branch James Russell, Tulsa, OK (US)

Correspondence Address: John E. Vick, Jr., Dority & Manning, Attorneys at Law, P.A., P.O. Box 1449, Greenville, SC, 29602, US

(21) Appl. No.: 09/852,978

(22) Filed: May 10, 2001

Related U.S. Application Data
(60) Provisional application No. 60/207,812, filed on May 30, 2000.

(51) Int. Cl. C07C 27/06

(52) U.S. Cl. 518/704

(57) ABSTRACT

Light hydrocarbons are converted into heavier hydrocarbons by preparing a synthesis gas, converting the synthesis gas to heavier hydrocarbons. Heat generated while preparing and converting synthesis gas is removed by creating steam that is then used in a water treatment unit to treat a water stream to remove contaminants such as salt. A process for preparing heavier hydrocarbons from light hydrocarbons, electricity, and treated water uses the energy of the conversion process is used to power a electrical generator and the thermal energy is used to assist in treating the water. Systems to convert light hydrocarbons to heavier with water treatment and electrical production are also presented.

### CROSS-REFERENCE TO RELATED APPLICATIONS

This application claims priority to previously filed provisional Application No. 60/207,812 filed in the United States Patent and Trademark Office on May 30, 2000.

### FIELD OF THE INVENTION

The present invention relates to the conversion of hydrocarbons such as through the FischerTropsch reaction, and more particularly relates to a system and method for converting light hydrocarbons into heavier hydrocarbons and for treating water such as desalinating salt water.

### BACKGROUND OF THE INVENTION

The synthetic production of hydrocarbons by the catalytic reaction of synthesis gas is well known and is generally referred to as the Fischer-Tropsch reaction. The Fischer-Tropsch process was developed in early part of the Twentieth Century in Germany. It was practiced commercially in Germany during World War II and later in South Africa.

The Fischer-Tropsch reaction for converting synthesis gas (primarily CO and H₂) has been characterized by the following general reaction:

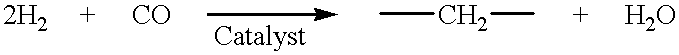

The hydrocarbon products derived from the Fischer-Tropsch reaction range from single carbon methane to higher molecular weight longer chained paraffinic waxes containing more than 100 carbon atoms.

Numerous Fischer-Tropsch catalysts have been used in carrying out the reaction, including cobalt, iron, and ruthenium, and both saturated and unsaturated hydrocarbons can be produced. The synthesis gas may be made from natural gas, gasified coal, and other sources. Three basic techniques have been employed for producing the synthesis gas (“syngas”), which is substantially carbon monoxide and molecular hydrogen. The three include oxidation, reforming, and autothermal reforming.

Fischer-Tropsch hydrocarbon conversion systems typically have a synthesis gas generator and a Fischer-Tropsch reactor unit. The synthesis gas generator receives light, short-chain hydrocarbons such as methane and produces synthesis gas. The synthesis gas is then delivered to a FischerTropsch reactor. In the F-T reactor, the synthesis gas is converted to heavier, longer-chain hydrocarbons. Recent examples of Fischer-Tropsch systems include U.S. Pat. Nos. 4,883,170; 4,973,453; 5,733,941; and 5,861,441 all of which are incorporated by reference herein for all purposes.

For water to be used as potable water or to adequately treat water to allow for its disposal in some instances, water is frequently treated. Such water treatments may involve boiling the water for set periods, aerating it, or removing salt and other contaminants. In certain parts of the world, the need to desalinate water is particularly valuable given the shortage of clean water. Through the application the term-contaminated water will be used to include salt water, brine, or other water with contaminates that are to be removed.

Salt water, sometimes referred to as “brine,” typically is desalinated by a thermal or membrane process. The thermal technique employs a distillation technique with the salt water being boiled and the resultant steam being collected and condensed into desalinated water. A widely used thermal process is the multistage flash distillation (or MSF) units. In a MSF unit, the heated salt water is fed into a flash chamber in which the pressure if lowered to allow the salt water to boil at lower temperatures. The resultant steam is condensed on tubes that carry the incoming salt water into the system. The steam heats the cooler incoming salt water and the vapor condenses to form desalinate. The remaining salt water, which is now more concentrated, goes to a second chamber that is at a lower pressure and the steam/condensation process is repeated there. Numerous chambers may be used in a plant.

Because of the need to avoid the formation of calcium sulphate salts through precipitation on surfaces in the water treatment facility, the temperature of the boiling salt water is limited to about 120 C. This typically increases the energy requirement of the system. In the past, this energy has been provided in some circumstances by combining a desalination plant with an electrical power plant; see e.g., U.S. Pat. No. 5,346,592 and 5,622,605. Such systems usually take the steam from the power plant and use it as the thermal source for a MSF.

### SUMMARY OF THE INVENTION

Therefore, a need has arisen for a system and method that addresses shortcomings of prior systems and methods. According to an aspect of the present invention, a process for converting light hydrocarbons into heavier hydrocarbons and for treating water includes the steps of preparing a synthesis gas; converting the synthesis gas to heavier hydrocarbons; removing heat generated in the steps of preparing and converting synthesis gas by generating steam; treating a water stream to remove contaminants with a water treatment unit that uses thermal energy; and using the steam generated in the heat removal to provide thermal energy for the treating of the water.

According to another aspect of the present invention a system for converting light hydrocarbons into heavier hydrocarbons and for treating water that includes a hydrocarbon conversion system having a synthesis gas subsystem for receiving an oxygen-containing gas and light hydrocarbons and producing a synthesis gas, a synthesis subsystem coupled to the synthesis gas subsystem for receiving synthesis gas and converting the synthesis gas to heavier hydrocarbons, and wherein the hydrocarbon conversion system is operable to produce steam; and a water treatment subsystem coupled to the hydrocarbon conversion system for receiving thermal energy therefrom and using the thermal energy to treat water.

According to another aspect of the present invention, a process for converting light hydrocarbons into heavier hydrocarbons, treating water, and producing electricity includes the steps of converting light hydrocarbons into heavier hydrocarbons; using energy produced in the conversion to power an electrical generator; treating a water stream to remove contaminants; and using thermal energy from the conversion step to provide the thermal energy for the water treatment step.

The present invention provides numerous advantages and a number of examples follow. An advantage of the present invention is that the combination of a water treatment subsystem with a hydrocarbon conversion subsystem allows for greatly improved thermal efficiency of the combined system. Another advantage of the present invention is that the chemical process generating steam for this system generates more steam than co-generation systems by several orders of magnitude—in some embodiments, the quantity steam generated could power ten or more desalination units. Another advantage is that the quantity of water treated can be readily expanded by adding stages in one embodiment that uses an MSF. Another advantage is the system allows better economic performance when compared to a separate hydrocarbon conversion plant and a separate water treatment plant (e.g., desalination plant). As another advantage, the system and method are able to utilize the large volume of low-pressure steam produced by a Fischer-Tropsch conversion system.

### BRIEF DESCRIPTION OF THE DRAWINGS

For a more complete understanding of the present invention and advantages thereof, reference is now made to the following description taken in conjunction with the accompanying drawings in which like reference numbers indicate like features, and wherein:

FIG. 1 is a schematic diagram of one embodiment of a system according to an aspect of the present invention for converting hydrocarbons and treating water;

FIG. 2 is a schematic diagram of a second embodiment of a system according the present invention showing an integrated conversion system and water treatment system;

FIG. 3 is a schematic diagram of third embodiment of a system according to an aspect of the present invention for converting hydrocarbons and treating water;

FIGS. 4A and 4B present a schematic diagram of fourth embodiment of a system according to an aspect of the present invention for converting hydrocarbons and treating water; and

FIG. 5 presents an alternate embodiment of the system in which tail gas may be combusted to generate additional steam.

### DETAILED DESCRIPTION OF THE INVENTION

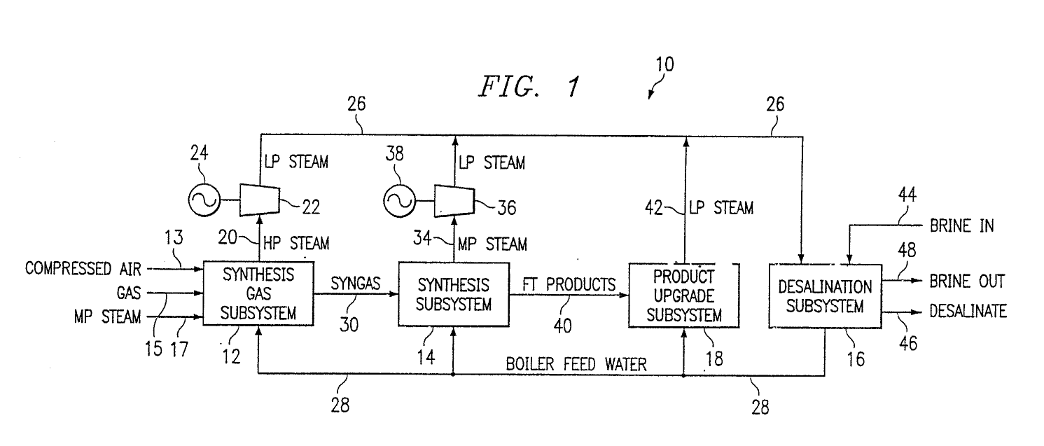

The present invention and its advantages are best understood by referring to FIGS. 1-5 of the drawings, like numerals being used for like and corresponding parts of the various drawings.

Referring to FIG. 1, a system 10 for converting light hydrocarbons into heavier hydrocarbons and treating water is presented. The water is treated to remove contaminates, such as salt, to produce a purified water. A prominent example used in the embodiments is to treat salt water to remove the salt. Fresh water may also be treated to remove other contaminants or with sufficient temperature to kill microorganisms.

The system 10 preferably includes a synthesis gas subsystem 12, a synthesis subsystem 14, and a water-treatment subsystem 16. The system 10 may further include a product upgrade subsystem 18. The synthesis gas subsystem 12 and synthesis subsystem 14 together form a hydrocarbon conversion system that takes light hydrocarbons and produces heavier hydrocarbons.

The synthesis gas subsystem 12 includes a synthesis-gas-generator. The synthesis gas generator (not explicitly shown) can take numerous forms such a partial oxidation unit, a catalytic partial oxidation unit, a steam methane reformer or an autothermal reformer (ATR), but is preferably an autothermal reformer unit with a quench cooler for the ATR exhaust. The synthesis gas subsystem 12 receives an oxygen-containing gas (e.g., air, oxygen, enriched air, etc., but preferably compressed air), light hydrocarbons, and steam through conduits 13, 15, and 17 and makes synthesis gas and makes a high pressure (on the order of about 600 psi) steam. The high-pressure steam is delivered by conduit 20 to turbine 22. Turbine 22 may power generator 24 or a synthesis gas booster compressor or some other item. The low-pressure steam (e.g., on the order of 25-50 psi) exiting the turbine 22 is delivered by conduit 26 to the water-treatment subsystem 16. The low-pressure steam of conduit 26 might be atmospheric or even a vacuum to provide for optimum temperatures in the heat exchangers associated with desalination subsystem 16 to prevent plating and other associate problems. Boiler feed water is delivered to the synthesis gas subsystem 12 through conduit 28. The synthesis gas generated in subsystem 12 is delivered by conduit 30 to synthesis subsystem 14.

The synthesis subsystem 14 receives the synthesis gas from conduit 30 and also boiler feed water from conduit 28. The boiler feed water is used to cool within subsystem 14 and it exits in conduit 34 as a medium pressure steam (preferably on the order of 150 psi) and is delivered—depending on pressure—to another turbine, a topping turbine 36. Turbine 36 can just be a stage of turbine 22. The turbine 36 may be used to drive a generator 38 or other equipment. The resultant low-pressure steam from turbine 36 is delivered to conduit 26 that delivers it to the water-treatment subsystem 16. The synthesis subsystem 14 receives the synthesis gas and makes Fischer-Tropsch products that are preferably delivered through conduit 40 to product upgrade subsystem 18.

The product upgrade subsystem 18 is used to form various products from the raw FT product such as by hydrogenating and hydrocracking. The product upgrade subsystem 18 generates considerable low-pressure steam (preferably on the order of about 50 psi) that is delivered through conduit 42 to conduit 26 and to water-treatment subsystem 16. The product upgrade system further processes the Fischer-Tropsch products for a variety of uses and/or stores the product.

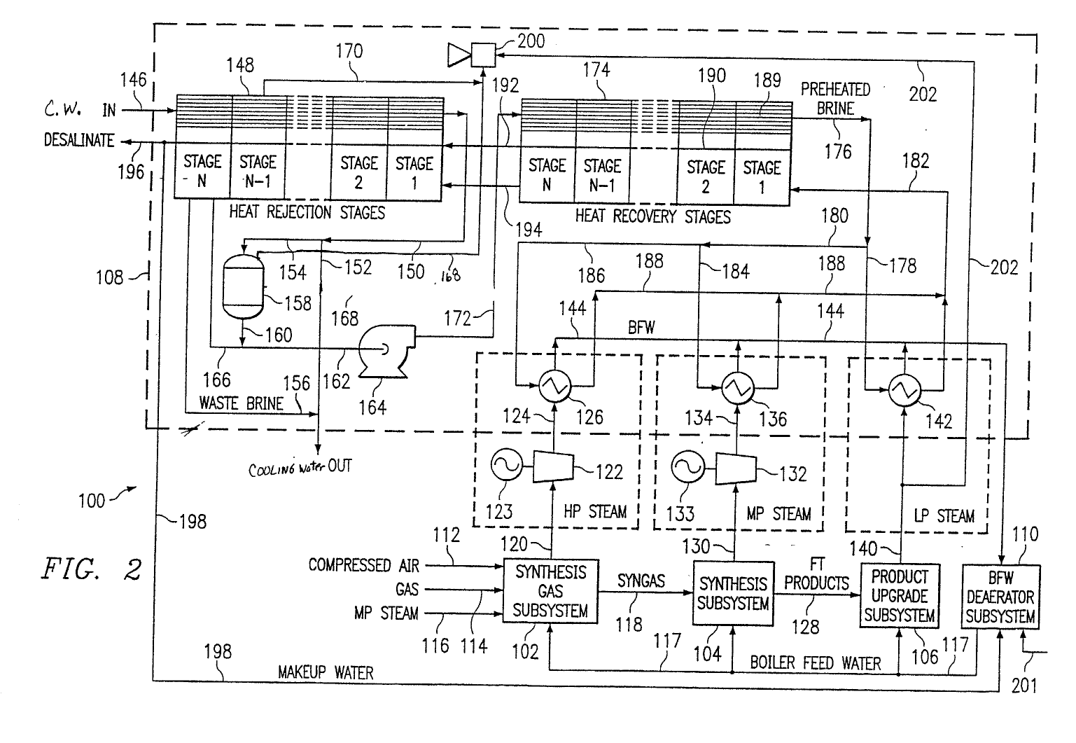

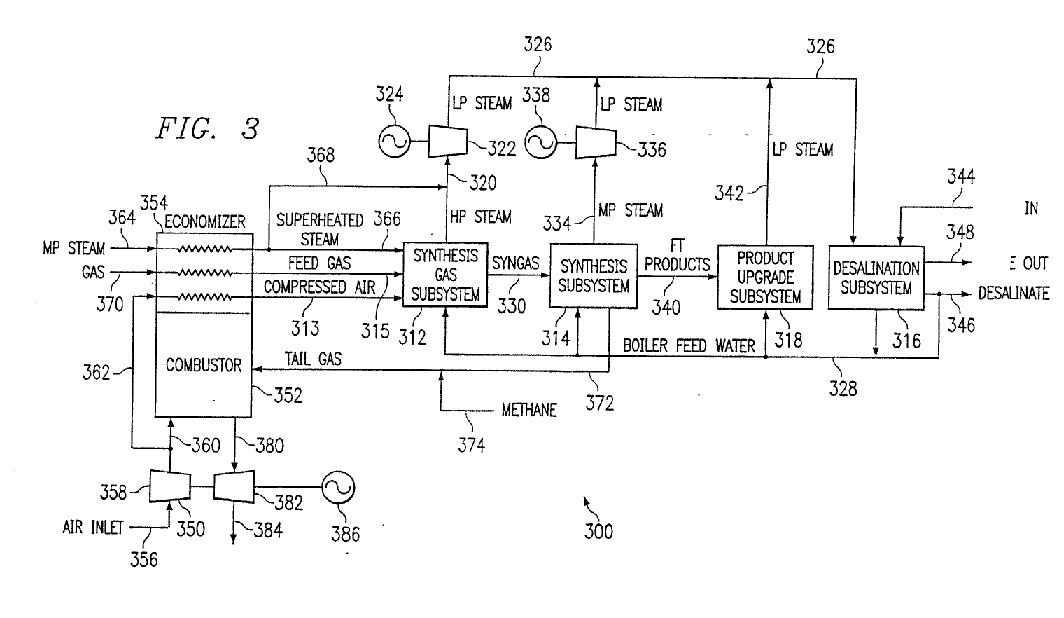

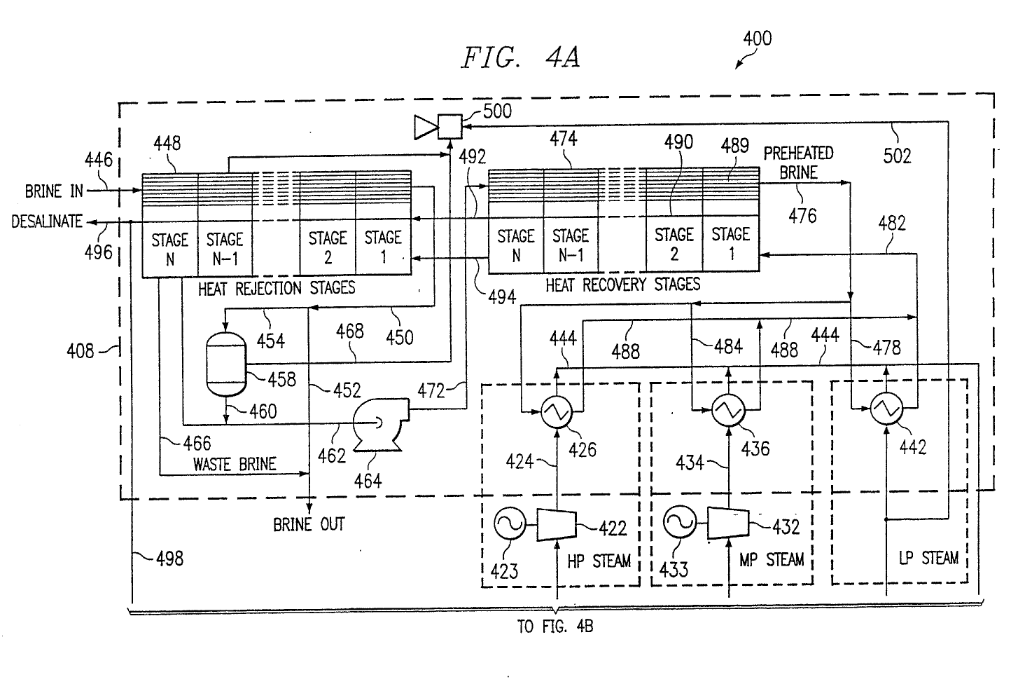

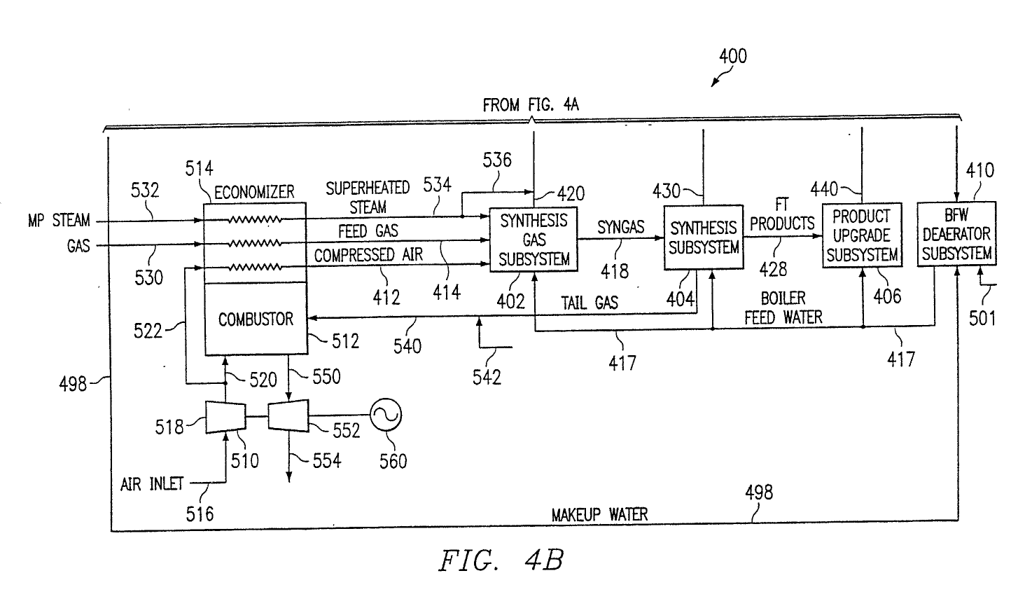

The thermal energy of the hydrocarbon conversion system (e.g. subsystems 12 and 14 and optionally 18) is used in the water treatment subsystem 16. In FIGS. 1 and 3, the thermal energy of the hydrocarbon conversion system is converted to steam and the steam used in water treatment subsystems. The thermal energy may also be used by transferring the thermal energy to water that is to be treated and sending the water to the water treatment subsystem as illustrated in the embodiments of FIGS. 2 and 4 which are described further below.

The water-treatment subsystem 16 receives the low-pressure steam through conduit 26 and contaminated water to be treated, which may be a variety of types such as salt water in this example, through conduit 44. Then using any of a number of water-treatment techniques known in the art (e.g., for desalination, it may be a multistage flash (MSF) unit or multi-effect distillation (MED) unit or a combination unit), the subsystem 16 produces a treated water stream, which in this example is a desalinate, that exits through conduit 46. The desalinate may be chlorinated or otherwise treated before use as potable water. The non-desalinated contaminated water exits through conduit 48. The non-desalinated, concentrated contaminated water of conduit 48 may be used in other systems or plants or may be exhausted back to sea or an ocean or other source assuming proper conditions. The boiler feed water exits through conduit 28 for use elsewhere in system 10. System 10 is essentially a “tri-Gen” plant in that it generates the Fischer-Tropsch product, electricity, and treated water.

Referring now to FIG. 2, another system 100 for converting light hydrocarbons into heavier hydrocarbons and desalinating contaminated water is presented. System 100 has a synthesis gas subsystem 102, a synthesis subsystem 104, a product upgrading subsystem 106, a desalination subsystem 108, and a boiler feed water deaerator subsystem 110.

The synthesis gas subsystem 102 receives an oxygen-containing gas, light hydrocarbons (e.g., methane), and steam through conduits 112, 114, and 116, respectively. The subsystem 102 produces synthesis gas that is delivered through conduit 118 to synthesis subsystem 104 and a high-pressure steam that is delivered to conduit 120. Boiler feed water is delivered to synthesis gas subsystem 102 through conduit 117 and used to cool with high-pressure steam being generated. The high-pressure steam is delivered through conduit 120 to a turbine 122 that is driven thereby and from which boiler feed water exits having been condensed. Turbine 122 may drive a generator 123 or be used for shaft horsepower for another purpose. The expanded steam/water exists and is condensed by exchanger 126 and the resultant boiler feed water is delivered to conduit 124 and then to conduit 144. As will be described further below, the boiler feed water exiting the turbine 122 is used to further heat pre-heated contaminated water to at or near its flash point temperature in heat exchanger 126 although it does not flash there because it is under pressure.

The synthesis subsystem 104 receives the synthesis gas 118 and preferably produces Fischer-Tropsch products that are delivered through conduit 128 to storage or to the product upgrading subsystem 106. Boiler feed water is supplied through conduit 117 to subsystem 104 for cooling and a medium pressure steam in generated thereby. The medium pressure steam is delivered through conduit 130 to turbine 132. The turbine 132 typically is used to drive a generator 133 or other device. The steam/water is delivered to heat exchanger 136 and the resultant condensed boiler feed water is delivered into a portion conduit 134 and then to conduit 144. Conduit 134 includes the heat exchanger 136 that is used for further heating pre-heated contaminated water to at or near its flash point (although it does not flash because its under pressure) as will be described further below. The heat exchangers 126 and 136 are shown as separate devices from the turbines 122 and 132, but can be an integral part of the turbine itself as many designs are possible; this is suggested in the drawing by the broken-line boxes around the turbines and heat exchangers.

The Fischer-Tropsch (“FT”) products are delivered through conduit 128 to the product upgrading subsystem 106. Subsystem 106 takes the raw Fischer-Tropsch products and modifies them into various desirable products. The subsystem 106 receives boiler feed water through conduit 117 and produces large quantities of low-pressure steam that is delivered to conduit 140. Conduit 140 contains a heat exchanger 142 that is used to further heat pre-heated contaminated water to at or near its flashing point, although it does not flash because it is under pressure as will be described further below. Conduits 124, 134, and 140 deliver boiler feed water to conduit 144.

Conduit 144 delivers the water to boiler feed water deaerator subsystem 110. After deaerating the water, the deaerator subsystem 110 delivers the boiler feed water to conduit 117 for use in the system as previously described. Process water from the synthesis gas subsystem or synthesis subsystem may be delivered through conduit 201 to deaerator subsystem 110.

Referring now to the de-salination subsystem 108, contaminated water is introduced through conduit 146 to a heat rejection portion or stages 148 of subsystem 108. Stages 1 through N are shown for heat rejection portion 148. The contaminated water rejects heat as it travels through portion 148 and then is delivered to conduit 150, which connects with conduit 152 and 154. The contaminated water in conduit 152 is combined with waste contaminated water delivered through conduit 156 before exiting subsystem 108. The salinity of the waste contaminated water in conduit 156 is adjusted to be safe for disposal in any oceans, lakes or seas. A side stream is pulled off of the contaminated water in conduit 146 and delivered by conduit 154 to a chemical injection drum 158 before being delivered by conduits 160 and 162 to contaminated water pump 164. Drum 158 serves as a deaerator and allows injection of pretreatment chemicals. Conduit 166/172 is a contaminated water recycle. Conduit 168 maintains a vacuum. Contaminated water pump 164 motivates the contaminated water through conduit 172 to the heat recovery portion or stages 174. Conduit 170 allows a vacuum to be pulled on all stages and removes non-condensables.

Heat is recovered by the contaminated water delivered by conduit 172 in stages 174 such that preheated contaminated water is delivered into conduit 176. Conduit 176 delivers the pre-heated contaminated water to heat exchanger 142 and then to conduit 182. Conduit 180 delivers pre-heated contaminated water to conduits 184 and 186. Conduit 184 delivers the heated contaminated water to heat exchanger 136 and then to conduit 188. The contaminated water in conduit 186 is delivered through heat exchanger 126 to conduit 188. Conduit 188 delivers the heated contaminated water to conduit 182. Generally, it is desirable to keep the contaminated water delivered by conduit 182 below 195° F. to avoid problems with precipitates in the contaminated water. Hotter temperatures are possible but more frequent cleaning and chemicals will be required. Conduit 182 delivers the heated contaminated water to heat recovery portion 174. Heat exchangers 126, 136, and 142 further heat the preheated contaminated water to the point that the contaminated water is ready to flash once the pressure is reduced at the first stage of the heat recovery portion 174. The first stage is at a reduced pressure (sub-atmospheric) such that the contaminated water delivered by conduit 182 flashes, and stage 2 is at still a further reduced pressures such that the contaminated water again flashes and so forth to stage N. The contaminated water is evaporated and then condensed in heat recovery stages 174. The contaminated water flashes and gets condensed on coils 189 and collected in the trough 190. The desalinate in trough 190 migrates towards conduit 196 because of the pressure gradient between stages.

The desalinate is shown continuing between the heat recovery portion 174 and the heat rejection portion 148 by conduit 192 and similarly the contaminated water is transported between stages by conduit 194. Large quantities of contaminated water are brought through conduit 146 to provide a one pass cooling of the contaminated water delivered through conduit 194 to the heat rejection stage 148 before the waste contaminated water is removed from system. 108. Upon reaching the n-th stage of the heat rejection portion 148, the wasted contaminated water is delivered to conduit 156. The cumulative desalinate is delivered to conduit 196 from it where it may be used for any purpose desired for the desalinated water. A portion of the desalinate is removed from conduit 196 by conduit 198 to be used a make-up water for other portions of system 100. The water in conduit 198 is delivered to the boiler feed water deaerator subsystem 110.

Steam eductor 200 receives low pressure or medium pressure steam through conduit 202 which receives the steam from conduit 140. Steam eductor 200 also receives inputs from conduits 170 and 168. Steam eductor 200 is used to adjust the pressures within the various stages and devices of desalination subsystem 108 and to remove non-condensables.

Referring now to FIG. 3, a system 300 for converting light hydrocarbons into heavier hydrocarbons and for desalinating sea water is presented. System 300 is analogous in most respects to system 10 of FIG. 1, but notably has the addition of turbine 350 having combustor 352 and economizer or heat recovery steam generating (HRSG) unit 354 added on a front portion. In another embodiment, a gasifification unit could be substituted for combustor 352.

Turbine 350 receives an oxygen-containing gas, preferably air, through inlet or intake conduit 356 which is compressed by compressor section 358 of turbine 350. The compressed air is delivered by conduit 360 to combustor 352, but a portion of the compressed air is removed from conduit 360 and delivered by conduit 362 to conduit 313 after passing through economizer 354. Economizer 354 is associated with combustor 352 and is used to recover heat therefrom. Thus the compressed air of conduit 313 is compressed heated air that is delivered to the synthesis gas subsystem 312. The economizer 354 also receives medium pressure steam through conduit 364 and super heats it before delivery to conduit 366. If any super heated steam in conduit 366 is not needed for the synthesis gas subsystem 312, it is delivered by conduit 368 to the high pressure steam of conduit 320. Light hydrocarbons such as natural gas are delivered by conduit 370 to economizer 354 where the gas stream is heated and then delivered to conduit 315.

Combustor 352 preferably is fueled by a low-BTU tail gas (for example 100 BTU/cu. ft. or less) that is delivered through conduit 372 after having been generated in the synthesis subsystem 314. The BTU content of the tail gas in conduit 372 can also be higher than 100 BTU and further in some instances it may be desirable to further enrich BTU content by adding methane or other enriching gases through conduit 374. The gas delivered by conduit 372 to combustor 352 is combusted and the exhaust is delivered through conduit 380 to expansion section 382 of turbine 350 and then exhausted through exhaust conduit or outlet 384. Turbine 350 may be used to drive a generator 386 or other device.

Referring now to FIG. 4, another embodiment of a system 400 for converting light hydrocarbons into heavier hydrocarbons and for desalinating salt water is presented. System 400 is analogous to system 100 of FIG. 2 in most respects, but notably, turbine 510 with associated combustor 512 and associated economizer or heat recovery steam generator 514 have been added on a front portion. To conveniently present the analogous components and aspects of FIG. 4 as compared to FIG. 2, corresponding parts and subsystems have been identified with reference numerals related in that FIG. 2 starts with reference numeral 100 and carries through reference numeral 202 and the corresponding parts of FIG. 4 begin with reference numeral 400 and carry through reference number 502 with the last two digits being identical for corresponding parts.

An oxygen-containing gas is supplied to inlet or conduit 516 that is compressed in compressor section 518 of turbine 510. The compressed gas is delivered at least in part by conduit 520 to combustor 512. A portion of the compressed gas in conduit 520 is removed by conduit 522 for use in the hydrocarbon conversion subsystems. The compressed gas of conduit 522 is heated in economizer 514 to supply compressed heated air (or other oxygen containing gas) to conduit 412. Light hydrocarbons such as natural gas are supplied through conduit 530 to economizer 514 where the gas is heated and then delivered to conduit 514.

Steam, preferably a medium-pressure steam, is delivered to conduit 532 which is then delivered through economizer 514 where super heated steam is produced and delivered to conduit 534. The super heated steam is delivered to synthesis gas subsystem 402, but if not all of the super heated steam is needed, the excess is delivered by conduit 536 into conduit 420. Synthesis subsystem 404 produces a Fischer-Tropsch product stream delivered to conduit 428, but also a tail gas such as a low BTU tail gas (less than 100 BTU/cu. ft.) that is delivered to conduit 540 which in turn delivers the gas to combustor 512. A richer BTU content tail gas may be used and in addition in some instances it may be desirable to enrich the tail gas by adding a fuel gas such as methane through conduit 542. The exhaust products of combustor 512 are delivered by conduit or inlet 550 to the expansion section 552 of turbine 510 and then exhausted through outlet or conduit 554. Turbine 510 may drive generator 560 or other devices.

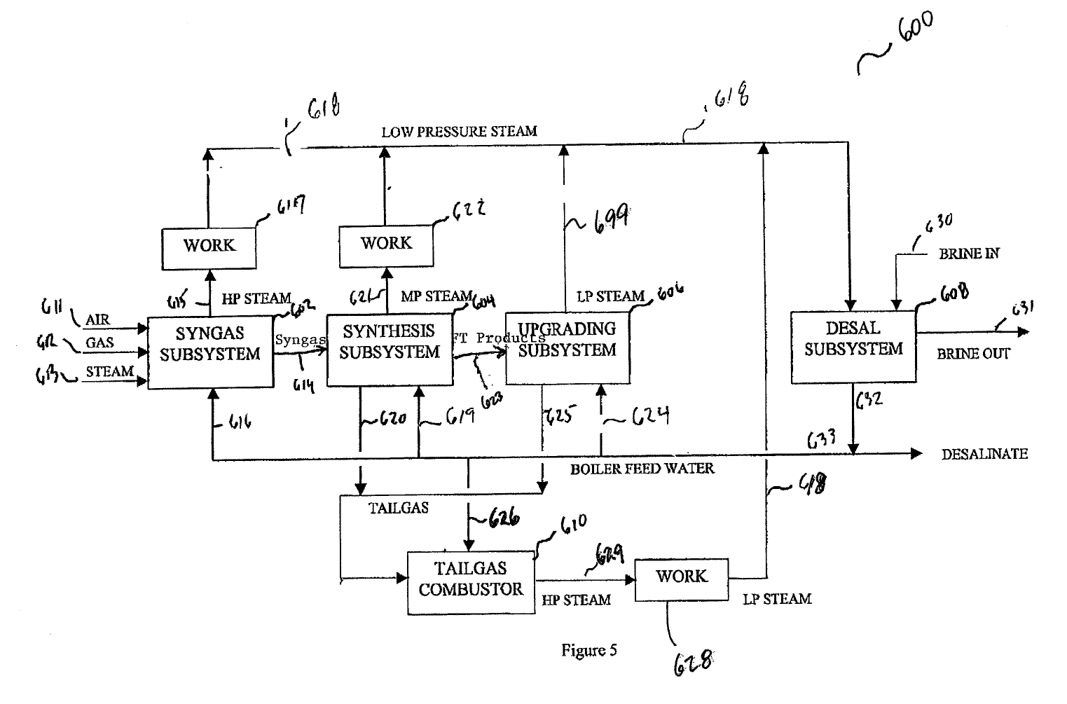

In FIG. 5, an additional embodiment of the invention is shown in which all or part of the tail gas may be combusted in a relatively low BTU combustor to generate additional steam in the system. In this embodiment, the combustor takes on the form of a steam boiler, which may be fitted with low BTU burners. The low BTU combustor may burn pure tail gas or a mixture of tail gas and other higher BTU fuels. The higher BTU fuel may be blended with the tail gas, burned in dedicated burners, or a combination of the two methods may be used.

The system 600 includes a synthesis gas (“syngas”) subsystem 602, a synthesis subsystem 604, a product upgrading subsystem 606, a desalination subsystem 608 and a tail gas combustor 610.

The synthesis gas subsystem 602 receives an oxygen-containing gas, light hydrocarbons (e.g., methane), and steam through conduits, 611, 612, and 613 respectively. The subsystem 602 produces synthesis gas that is delivered through conduit 614 to the synthesis (syngas) subsystem 604 and high pressure steam that is delivered to conduit 615. Boiler feed water is delivered to synthesis gas (syngas) subsystem 602 through conduit 616 and is used for cooling in the subsystem, with high pressure steam being produced. The high pressure steam is delivered through conduit 615 to a module 617 that is used to extract work from the steam. The work module 617 may take on the form of a steam turbine that in turn is used to drive an electrical power generator, a compressor, or in other cases is another piece of equipment designed to perform other mechanical work. The steam may also be used for other types of work such as heating/cooling or other thermal or mechanical processes. In the process of performing work the high pressure steam is converted to low pressure steam and/or condensate which is delivered from the work module 617 to the desalination subsystem 608 through conduit 618.

The synthesis subsystem 604 receives the synthesis gas from conduit 614 and also receives boiler feed water through conduit 619. The boiler feed water is used for cooling within the subsystem with medium pressure steam being produced. The medium pressure steam (preferably on the order of 150 psi), is delivered to work module 622 through conduit 621. Work module 622 can take on the form of a steam turbine for producing mechanical shaft work such as work module 617 previously described or can in fact form a part of work module 617 by injecting the lower pressure steam into a suitable stage of a larger steam turbine. The medium pressure steam can also be used for other thermal work such as heating or cooling and to supply other process needs such as the steam required by the synthesis gas module 602. The resultant low pressure steam is delivered to conduit 618 which then delivers it to the water treatment subsystem 608. The synthesis subsystem 604 receives synthesis gas and makes Fischer-Tropsch products that are preferably delivered through conduit 623 to product upgrading subsystem 606. Synthesis subsystem 604 produces a low BTU tailgas (preferably on the order of 60-100 BTU/SCF) and delivers it to low BTU combustor 610 through conduit 620 where it is combusted. It should also be noted that the synthesis subsystem 604 can be operated in a multitude of ways so as to produce tailgas that can range in heat content from 50 BTU/SCF or below to 300 BTU/SCF or above. This may be advantageous under certain economic conditions in which there is little or no monetary value placed on certain Fischer-Tropsch products but there is a relatively large value placed on the work that can be realized from steam generation (e.g., electrical power generation or additional treated water capacity).

Fischer-Tropsch products are delivered through conduit 623 to product upgrading subsystem 606. Subsystem 606 takes the raw Fischer-Tropsch products and modifies them to various desirable products. Subsystem 606 receives boiler feed water through conduit 624 and produces low pressure steam that is delivered to the water treating subsystem through line 699 and on through conduit 618. Subsystem 606 also produces tailgas that is relatively small in amount but relatively high in BTU content when compared to tailgas produced in the synthesis section. This tailgas is preferably blended with the low BTU tailgas produced in subsystem 604 and delivered to the tailgas combustor 610 through conduit 625.

Tailgas combustor 610 receives boiler feed water through conduit 626 and receives tailgas through conduits 620 and/or 625. The tailgas is combusted to and the resulting energy release may be used to raise high pressure steam which is delivered to work module 628 through conduit 629. Work module 628 extracts work from the steam in a similar manner and by a similar variety of mechanisms as do work modules 617 and 622 previously described. The resultant low pressure steam is delivered to water treatment subsystem 608 through conduit 618. In addition to steam generation, there are a number of other methods by which the heat generated by combusting the tailgas can be used. This includes superheating steam, and/or preheating the oxygen-containing as and light hydrocarbon streams used in synthesis gas subsystem 602 or other process heating purposes. By including multiple heating coils in tailgas combustor 610 it is possible to simultaneously provide several process heating services to system 600 in one device.

Desalination subsystem 608 receives contaminated water (i.e.: brine) through conduit 630 and low pressure steam through conduit 618. It produces purified water which is exported through conduit 632 and a portion of the produced water is made available to the various subsystems as boiler feed water through conduits 633, 616, 619, 626 and 624. The embodiment of the water treating system and various configurations thereof are as previously described.

In general, the hydrocarbon conversion and product upgrading aspects of the present invention may be used to make numerous longer-chain hydrocarbons, e.g., the full spectrum of C₅₊ products through the Fischer-Tropsch reaction (but other reactions might be used in some situations) and may be adapted to accommodate numerous environments and applications. The longer-chain Fischer-Tropsch products that may be made directly or with downstream processing include numerous products for numerous uses. A number of examples are presented below.

The Fischer-Tropsch products may include synthetic alpha olefins adapted for many applications, including, without limitation, PAO feedstock (alpha olefins in the range of C₆ to C₁₂ and preferably C₁₀ are used to produce poly alpha olefins); alpha olefins for laundry and other detergents (preferably C₁₂-C₁₆); chlorination stock to be used in textiles, pharmaceuticals and transportation lubricants/hydraulic fluids (preferably C₁₈-C₂₄); alpha olefins used to produce particle board emulsions and poly vinyl chloride lubricants (C₂₄-C₂₈); and alpha olefins used to manufacture decorative and industrial candles, particle board emulsions and PVC lubricants (C₃₀+ alpha olefins, which are considered a synthetic paraffin wax and therefore used in many of the markets where paraffin waxes are used). The Fischer-Tropsch products are also well suited for use as a synthetic white oils because Fischer-Tropsch liquid normal paraffins meet FDA specifications governing their use in direct food contact applications, which gives them a wide range of potential markets to enter, including markets which traditionally use food grade mineral oils. Similarly, the Fischer-Tropsch products may be used for technical grade mineral or white oils that are used to produce paints, stains and inks, among other end-use products and may be used as a pharmaceutical (USP) grade white oil to be used to produce cosmetics and healthcare products. In these applications, Fischer-Tropsch products are better because the liquid or hydroisomerized product can probably satisfy ASTM standards with little effort.

The Fischer-Tropsch products may also be used for synthetic liquid n-paraffins in numerous applications. The Fischer-Tropsch product may be used as a chlorination feedstock to be used, for example, to produce chlorinated normal paraffins for use in textiles and industrial lubricants. The product may also be used as a linear alkyl benzene (LAB) feedstock (C₁₀ to C₁₃) which may be used for laundry detergents. The Fischer-Tropsch product may also be used as an aluminum rolling oil (C₁₄ to C₁₇), e.g., for cold rolling oils for aluminum foil. Further the Fischer-Tropsch product N-paraffin may be used for “liquid” candles.

The Fischer-Tropsch product may be used as a synthetic wax in numerous applications. For example, the product may be used to make thermostat wax, which is used primarily to control automobile thermostats. The wax is particularly suitable for this since it must be uniform in molecular weight, carbon number distribution and molecular structure. The Fischer-Tropsch wax may be used to make hotmelt adhesives, i.e., used as a viscosity modifier for industrial hotmelt adhesives. The synthetic wax may be used in printing inks . In that case, the wax is used as an antiscuff surface modifier for fine grade web offset and gravure inks. It may also be used for paints and stains. The wax is used to enhance water repellency of water-based paints and stains. The Fischer-Tropsch product may be used to make corrugated board in which the waxes are used to add strength and water repellency to the corrugated board. Similarly, the Fischer-Tropsch product may also be used as a wax for packaging and food additives.

The synthetic wax may be used as a PVC lubricant/extrusion aid; the high melting point waxes are used as internal/external lubricants for PVC extrusion. The wax may be used as a flushing compound, to impart the dripless quality to decorative candles, with cosmetics as a viscosity modifier and melting point enhancer, to bind various drugs which are in powdered form into tablet form (they also impart a slippery surface to tablets such as aspirin, etc.). Waxy Fischer-Tropsch products may also be used as plasticizers and extrusion aids for various plastics such as high density polyethylene, PET linear low density polyethylene and polypropylene. Another use is as anti-ozone additives to protect the outside surfaces of rubber products from packing and ozone damages.

Fischer-Tropsch product in the form of synthetic lubricants may be used in numerous additional applications. For example, the synthetic lubricants may be used as environmentally friendly drilling fluids. Fischer-Tropsch oils may be used to produce highly stable high temperature operation automatic transmission fluids. They may also be used as a hydraulic fluid that is very stable at high temperatures and ideally suited for use in vehicular and industrial hydraulic compounds. The synthetic lubricants may also be used as vehicular lubricants (PCMO and HDD). The Fischer-Tropsch product in the form of a synthetic lubricant may be used as a quenching oil or cutting oil. Further they may be used for a plurality of specialty lubricants such as for two-cycle, marine lubricants, or baroil. They may also be used as a vehicle for lubricant-additives.

Products that may be made from or as part of the Fischer-Tropsch products are synthetic fuels and blends, including Fischer-Tropsch compression ignition fuels, Fischer-Tropsch spark ignition fuels, fuels for fuel cell systems, aviation fuel (turbine, spark-ignition, and compression5 ignition) and railroad fuels. The sulfur-free clean nature of the synthetic fuels thus made are advantageous. The Fischer-Tropsch products may also be used as synthetic solvents. As such, the uses of the synthetic solvents include as printing inks, paints, stains, drying agents, dye transfer agents, synthetic heptane, hexane, and de-waxing agents.

Although the present invention and its advantages have been described in detail, it should be understood that various changes, substitutions and alterations can be made therein without departing from the spirit and scope of invention as defined by the appended claims. For example, portions of one embodiment may be adapted and used with other suggested embodiments. Although the term desalination subsystem is used it is to be understood that it encompasses the broader water treatment system in that even waters from such sources as oil field waters could be cleaned up via these subsystems. While MSF units are presented for illustrative purposes other treatment subsystems may be used in a like manner such as a MED20 type desalination unit. Process water from the synthesis unit and synthesis gas unit may be used throughout the systems as well and may be treated by the water treatment system.

What is claimed is:

1. A process for converting light hydrocarbons into heavier hydrocarbons and for treating water, the process comprising the steps of:
preparing a synthesis gas;
converting the synthesis gas to heavier hydrocarbons having at least about five or more carbon atoms per molecule;
removing heat generated in the steps of preparing synthesis gas by generating steam;
treating a water stream to remove contaminants with a unit using thermal energy; and
using the steam generated in the heat removal to provide thermal energy for treating the water.

2. The process of claim 1 wherein the step of preparing a synthesis gas comprises:
delivering a compressed oxygen-containing gas to a synthesis-gas-generator;
delivering a light hydrocarbon stream to the synthesis-gas-generator;
delivering steam to the synthesis-gas-generator; and reacting the oxygen-containing gas, light hydrocarbon stream, and steam in the synthesis-gas-generator to produce synthesis gas.

3. The process of claim 1 wherein the step of preparing a synthesis gas comprises:
delivering a compressed oxygen-containing gas to an autothermal reformer;
delivering a light hydrocarbon stream to the autothermal reformer;
delivering steam to the synthesis-gas-generator; and reacting the oxygen-containing gas, light hydrocarbon stream, and steam in the autothermal reformer to produce synthesis gas.

4. The process of claim 1 wherein the step of converting the synthesis gas to heavier hydrocarbons comprises:
delivering the synthesis gas to a Fischer-Tropsch reactor; and
reacting the synthesis gas with a Fischer-Tropsch catalyst to produce the heavier hydrocarbons.

5. The process of claim 1 wherein the steps of treating a water stream and using the thermal energy, comprises the steps of:
preheating a contaminated water stream;
further heating the contaminated water stream with thermal energy from the conversion of synthesis gas into heavier hydrocarbons;
flashing the heated contaminated water of the previous step in a plurality of stages to create a purified water;
collecting the purified water; and
disposing of the remaining untreated water.

6. The process of claim 1 wherein the steps of preparing a synthesis gas and converting the synthesis gas comprise the steps of:
delivering an oxygen-containing gas to a compressor section of a turbine;
compressing the oxygen-containing gas and delivering it to an economizer associated with a combustor of the gas turbine;
delivering light hydrocarbons and steam to the economizer;
heating the steam, gas, and compressed oxygen-containing gas in the economizer;
delivering the heated steam, gas, and compressed-oxygen containing gas to an synthesis gas unit wherein synthesis gas is generated;
delivering the synthesis gas to a synthesis unit wherein the synthesis gas is converted to heavier hydrocarbons having at least about five carbon atoms per molecule and a low-BTU tail gas;
delivering the low-BTU tail gas to the combustor of the gas turbine for use as a fuel therein; and
using boiler feed water to remove thermal energy from the synthesis gas unit and the synthesis unit.

7. The process of claim 1 wherein:
(a) the steps of preparing a synthesis gas and converting the synthesis gas comprise the steps of:
delivering an oxygen-containing gas to a compressor section of a
turbine, and then compressing the oxygen-containing gas and delivering it to an economizer associated with a combustor of the gas turbine,
delivering light hydrocarbons and steam to the economizer, heating the steam, gas, and compressed oxygen-containing gas in the economizer,
delivering the heated steam, gas, and compressed-oxygen containing gas to an synthesis gas unit wherein synthesis gas is generated,
delivering the synthesis gas to a synthesis unit wherein the synthesis gas is converted to heavier hydrocarbons having at least about five carbon atoms in chain length, and a low-BTU tail gas,
delivering the low-BTU tail gas to the combustor of the gas turbine for use as a fuel therein,
using boiler feed water to remove thermal energy from the synthesis gas subsystem and the synthesis unit; and
(b) the steps of treating a water stream and using the thermal energy, comprises the steps of:
preheating a contaminated water stream;
further heating the contaminated water stream with thermal energy from the conversion of synthesis gas into heavier hydrocarbons;
flashing the heated contaminated water of the previous step in a plurality of stages to create a purified water;
collecting the purified water; and disposing of the remaining untreated water.

8. A hydrocarbon product having a hydrocarbon molecule chain length of at least about five carbon atoms made from the process of claim 1.

9. A treated water stream made from the process of claim 1.

10. A process for converting light hydrocarbons into heavier hydrocarbons, treating water, and producing electricity, the process comprising the steps of:
converting light hydrocarbons into heavier hydrocarbons, wherein the step includes using a Fischer-Tropsch reaction;
using energy produced in the conversion to power an electrical generator;
treating a water stream to remove contaminants; and
using thermal energy from the conversion step to provide the thermal energy for the water treatment step.

11. A system for converting light hydrocarbons into heavier hydrocarbons and for treating water, the system comprising:
a hydrocarbon conversion system comprising:
a synthesis gas subsystem for receiving an oxygen-containing gas and light hydrocarbons and producing a synthesis gas,
a synthesis subsystem coupled to the synthesis gas subsystem for receiving synthesis gas and converting the synthesis gas to heavier hydrocarbons, and
wherein the hydrocarbon conversion system is operable to produce steam; and
a water treatment subsystem coupled to the hydrocarbon conversion system for receiving thermal energy therefrom and using the thermal energy to treat water.

12. The system of claim 1 wherein the water treatment subsystem comprises an MSF.

13. A process for converting light hydrocarbons into heavier hydrocarbons and for treating water, the process comprising the steps of:
(a) preparing a synthesis gas;
(b) converting the synthesis gas to heavier hydrocarbons having at least about five carbon atoms per molecule average;
(c) removing heat generated in the steps of preparing synthesis gas by generating steam;
(d) generating tail gas;
(e) combusing tail gas; and
(f) treating a water stream to remove contaminants with a unit using thermal energy.

14. The process of claim 13 in which heat generated by combusting the tailgas in step (e) is employed in part for superheating steam.

15. The process of claim 13 in which heat generated by combusting the tailgas in step (e) is employed in part for preheating an oxygen-containing gas.

16. The process of claim 13 in which heat generated by combusting the tailgas in step (e) is employed in part for preheating a light hydrocarbon stream.
